(19) United States
(12) Patent Application Publication
(10) Pub. No.: US 2023/0126071 A1
(43) Pub. Date: Apr. 27, 2023

(54) SYSTEMS AND METHODS FOR PRODUCING MIXTURES

(71) Applicants: Boston Scientific Medical Device Limited, Galway (IE); Boston Scientific Scimed, Inc., Maple Grove, MN (US)

(72) Inventors: Benjamin CLEVELAND, Bellingham, MA (US); Nitesh Ghananil BAVISKAR, Kalyan West (IN); Junaid Mohammed SHAIKH, Surat (IN); Richard Earl GRAFFAM, Pelham, NH (US); Joseph HERNANDEZ, Rutland, MA (US); Christopher WATSON, Lincoln, MA (US); Jennie CREEGAN, Minneapolis, MN (US); Kolbein KOLSTE, Acton, MA (US); Nicholas DeSANTIS, Cambridge, MA (US); Eric KIM, Brighton, MA (US); Mei Lee AMEND, Lakeville, MA (US); Subodh MOREY, Ponda (IN)

(73) Assignees: Boston Scientific Medical Device Limited, Galway (IE); Boston Scientific Scimed, Inc., Maple Grove, MN (US)

(21) Appl. No.: 18/048,546

(22) Filed: Oct. 21, 2022

Related U.S. Application Data
(60) Provisional application No. 63/262,952, filed on Oct. 22, 2021.

(51) Int. Cl. A61J 1/20

(52) U.S. Cl. CPC A61J 1/2089; A61J 1/202; A61J 1/201; A61J 1/2068

(57) ABSTRACT

A system is disclosed for producing a mixture to deliver to a treatment site. The system can include a mixing lumen attachable to a proximal end of a delivery system. A multi-lumen chamber can be connected to a proximal end of the mixing lumen and include a first lumen aligned and adjacent a second lumen. The first lumen can receive a first constituent in a first state. The first lumen can include a first plunger to move the first constituent from the first lumen to the mixing lumen. The second lumen can include a second constituent and a second plunger to distally move the second constituent and the first constituent in a second state. Distally moving the second plunger causes the first constituent and the second constituent to be mixed together within the mixing lumen to form the mixture.

### CROSS-REFERENCE TO RELATED APPLICATIONS

This patent application claims the benefit of priority under 35 U.S.C. § 119 to U.S. Provisional Patent Application No. 63/262,952, filed Oct. 22, 2021, which is herein incorporated by reference in its entirety.

### FIELD

The present disclosure relates generally to compositions for injection to a patient, methods of preparation and use thereof, and devices comprising such compositions.

### BACKGROUND

Numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy.

Since the conception of conformal radiotherapy, physicians have paid attention to the delivered dose to the target and surrounding tissues. Investigators have been able to correlate side effects to the amount of tissue receiving a certain radiation dose. And yet, time, distance, and shielding affect the dose that is delivered. The less time an area is exposed to radiation, the less dose delivered. The greater the distance from the radiation, the less dose delivered.

Current systems provide filler material to treatment sites to decrease the radiation dose to the rectum during radiotherapy for prostate cancer. However, the system that mixes the filler material in vitro includes numerous subcomponents, is complex to assemble, and rife with filler mixing errors prior to delivery within a patient at a treatment site. During the foregoing procedures, such errors and mishaps lead unnecessarily to patient risk, increased procedure time, and increased procedure costs. The solution of this disclosure resolves these and other issues of the art.

### SUMMARY

In accordance with certain aspects of the present disclosure, a system is disclosed for producing a mixture to deliver to a treatment site. A mixing lumen is attachable to a proximal end of a delivery system. A multi-lumen chamber can be connected to a proximal end of the mixing lumen. The multi-lumen chamber can include a first lumen aligned and adjacent a second lumen. The first lumen is configured to receive a first constituent in a first state. The first constituent is insertable through a first port into the first lumen. The first lumen can include a first plunger to move the first constituent from the first lumen to the mixing lumen. The second lumen is configured to include a second constituent, the second lumen including a second plunger to distally move the second constituent and the first constituent in a second state. The second lumen can terminate in a second port. In the second state, distally moving the second plunger causes the first constituent and the second constituent to be delivered through the first and second ports and mixed together within the mixing lumen to form the mixture.

In accordance with certain aspects of the present disclosure, the first port includes a luer fitting configured to receive a distal end of a syringe, and a distal end of the syringe and the first port are configured to removably connect with each other.

In accordance with certain aspects of the present disclosure, the first port is configured so that air can be purged from the first lumen through the first port.

In accordance with certain aspects of the present disclosure, the first constituent is a precursor mixture of a diluent with a hydrophilic polymer.

In accordance with certain aspects of the present disclosure, the first lumen includes a hydrophilic polymer prior to injection of the first constituent into the first lumen.

In accordance with certain aspects of the present disclosure, the hydrophilic polymer is polyethylene glycol.

In accordance with certain aspects of the present disclosure, the delivery system including a needle connected to a distal end of the mixing lumen.

In accordance with certain aspects of the present disclosure, injecting the first constituent through the first port into the first lumen by a syringe causes the first plunger to move proximally so the first constituent is received in the first lumen in the first state.

In accordance with certain aspects of the present disclosure, once the first and second constituents are within the first and second lumens, respectively, the second plunger is configured to distally move a first distance to purge air from the multi-lumen chamber.

In accordance with certain aspects of the present disclosure, the mixing lumen includes a static mixer.

In accordance with certain aspects of the present disclosure, at least one of the first port and the second port includes an air-permeable fluid-impermeable membrane.

In accordance with certain aspects of the present disclosure, the mixing lumen is included in a connector, the connector including a first asymmetric coupler positioned on a proximal end of the connector. The multi-lumen chamber includes a second asymmetric coupler positioned on a distal end of the multi-lumen chamber and configured to removably connect to the first asymmetric coupler.

In accordance with certain aspects of the present disclosure, the connector includes at or adjacent the first asymmetric coupler a first tube configured to be in fluid communication with the first port and the mixing lumen of the connector in the second state; and a second tube configured to be in fluid communication with the second port and the mixing lumen of the connector in the second state.

In accordance with certain aspects of the present disclosure, the first asymmetric coupler includes one or more latches configured to removably connect with one or more latches of the second asymmetric coupler. At least one of the first and second asymmetric coupler includes an actuator positioned on an outer surface configured to be squeezed so as to release a coupling between the one or more latches of the first and second asymmetric couplers.

In accordance with certain aspects of the present disclosure, the first and second asymmetric couplers each include an asymmetric outer surface profile shaped as a mirror of the other.

In accordance with certain aspects of the present disclosure, a method is disclosed for producing a mixture by a mixing system. The mixing system can include a multi-lumen chamber connected to a proximal end of a mixing lumen. The multi-lumen chamber can include a first lumen aligned and adjacent a second lumen. The first lumen can be configured to receive a first constituent in a first state. The first constituent can be insertable through a first port into the first lumen. The first lumen can include a first plunger to move the first constituent from the first lumen to the mixing lumen. The second lumen can include a second constituent, a second plunger, and the second lumen terminating in a second port. The method can include moving the first constituent from the first lumen to the mixing lumen by the first plunger; distally moving the second constituent and the first constituent by the second plunger; and distally moving the second plunger thereby causing the first constituent and the second constituent to be delivered through the first and second ports and mixed together within the mixing lumen to form the mixture.

In accordance with certain aspects of the present disclosure, the first port includes a luer fitting configured to receive a distal end of a syringe, and wherein a distal end of the syringe and the first port are configured to removably connect with each other.

In accordance with certain aspects of the present disclosure, the method can include purging air from the first and second lumens by distally moving the second plunger a first distance before forming the mixture.

In accordance with certain aspects of the present disclosure, the step of purging includes egressing air through an air-permeable fluid-impermeable membrane positioned on at least one of the first port and the second port.

In accordance with certain aspects of the present disclosure, the mixing lumen is included in a connector including a first asymmetric coupler positioned on a proximal end of the connector. The multi-lumen chamber includes a second asymmetric coupler positioned on a distal end of the multi-lumen chamber and configured to removably connect to the first asymmetric coupler. The first asymmetric coupler includes one or more latches configured to removably connect with one or more latches of the second asymmetric coupler. At least one of the first and second asymmetric coupler include an actuator positioned on an outer surface configured to be squeezed so as to release a coupling between the one or more latches of the first and second asymmetric couplers.

In accordance with certain aspects of the present disclosure, the mixing lumen is included in a connector including a first asymmetric coupler positioned on a proximal end of the connector. The multi-lumen chamber includes a second asymmetric coupler positioned on a distal end of the multi-lumen chamber and is configured to removably connect to the first asymmetric coupler. The first and second asymmetric couplers each include an asymmetric outer surface profile shaped as a mirror of the other.

In accordance with certain aspects of the present disclosure, the method can include connecting a primed connector and a delivery system to an asymmetric coupler of the multi-lumen chamber, the primed connector including the mixing lumen.

In accordance with certain aspects of the present disclosure, the method can include positioning a hydrophilic polymer in the first lumen prior to injection of the first constituent into the first lumen.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE FIGURES

The accompanying drawings, which are incorporated in and constitute a part of this specification, illustrate various exemplary aspects of the disclosure, and together with the description serve to explain the principles of the present disclosure.

FIGS. 1A-1B depict the prostate, rectum, and Denonvilliers' space between the prostate and rectum.

FIG. 2 shows an exploded view of an exemplary mixing system in accordance with certain aspects of the present disclosure.

FIG. 3 shows an exploded view of an exemplary mixing system in accordance with certain aspects of the present disclosure.

FIG. 4 depicts a partial cross-section view of an example connector and needle attached to an example mixing system, in accordance with certain aspects of the present disclosure.

FIG. 5A depicts an example step in a method using an example mixing system, in accordance with certain aspects of the present disclosure.

FIG. 5B depicts an example step in a method using an example mixing system, in accordance with certain aspects of the present disclosure.

FIG. 6A depicts an example step in a method using an example mixing system, in accordance with certain aspects of the present disclosure.

FIG. 6B depicts an example step in a method using an example mixing system, in accordance with certain aspects of the present disclosure.

FIG. 7 depicts an example step in a method using an example mixing system, in accordance with certain aspects of the present disclosure.

FIG. 8A depicts an example step in a method using an example mixing system, in accordance with certain aspects of the present disclosure.

FIG. 8B depicts an example step in a method using an example mixing system, in accordance with certain aspects of the present disclosure.

FIGS. 9A-9C depict example steps in a method of priming an example connector, in accordance with certain aspects of the present disclosure.

FIG. 10A depicts an exemplary mixing system in accordance with certain aspects of the present disclosure.

FIGS. 10B depicts a partial cross-section view of the system of FIG. 10A, in accordance with certain aspects of the present disclosure.

FIGS. 11A-11C depicts example steps in a method using the example mixing system of FIG. 10A, in accordance with certain aspects of the present disclosure.

FIGS. 12A-12C depicts example steps in a method using the example mixing system of FIG. 10A, in accordance with certain aspects of the present disclosure.

FIG. 13A depicts an exemplary mixing system in accordance with certain aspects of the present disclosure.

FIGS. 13B depicts a partial close-up view of the system of FIG. 13A, in accordance with certain aspects of the present disclosure.

FIGS. 13C depicts a partial close-up view of the system of FIG. 13A, in accordance with certain aspects of the present disclosure.

FIG. 14 depicts a flow diagram of a method of using a mixing system according to certain aspects of this disclosure.

### DETAILED DESCRIPTION

Particular aspects of the present disclosure are described in greater detail below. The terms and definitions provided herein control, if in conflict with terms and/or definitions incorporated by reference.

Particular aspects of the present disclosure may repeat reference numerals and/or letters in the various examples. This repetition is for the purpose of simplicity and clarity and does not in itself dictate a relationship between the various embodiments and/or configurations discussed. Different embodiments may have different advantages, and no particular advantage is necessarily required of any embodiment.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise.

As used herein, “approximately” and “about” refer to being nearly the same as a referenced number or value. As used herein, the terms “approximately” and “about” should be understood to encompass ±10% of a specified amount or value (e.g., “about 90%” can refer to the range of values from 81% to 99%).

As used herein, “operator” can include a doctor, surgeon, or any other individual or delivery instrumentation associated with delivery or use of a mixing system as such systems are described throughout this disclosure.

The compositions herein may be used in various medical procedures, including but not limited to injected to create additional space between the rectum and prostate during treatment, for example in the Denonvilliers' space, thereby reducing rectal radiation dose and associated side effects. Certain embodiments of the disclosure include placing a filler between the radiation target tissue and other tissues. The filler can be a gel composition that increases the distance between the target tissue and other tissues so that the other tissues receive less radiation.

It is understood that “Denonvilliers' space” is a region located between the rectum and prostate. Certain embodiments provide a method of displacing a tissue to protect the tissue against the effects of a treatment involving radiation or cryotherapy. One embodiment involves using a filler mixed by a mixing system of this disclosure to displace the tissue relative to a tissue that is to receive the treatment. Another embodiment involves introducing a filler mixed by a mixing system of this disclosure to displace a first tissue and radiating a second tissue, particularly a second tissue that is close to the first tissue. In another embodiment, the method includes the steps of injecting a filler into a space between tissues; and may further include irradiating one of the tissues so that the other tissue receives less radiation than it would have in the absence of the filler.

Certain embodiments also provide methods for treating a tissue of a body by radiation. In one embodiment, the method includes the steps of injecting an effective amount of a filler into a space between a first tissue (e.g., prostate) of a body and a second tissue (e.g., rectum), which can be a critically sensitive organ; and treating the first tissue by radiation whereby the filler within the space reduces passage of radiation into the second tissue. Tissue is a broad term that encompasses a portion of a body: for example, a group of cells, a group of cells and interstitial matter, an organ, a portion of an organ, or an anatomical portion of a body, e.g., a rectum, ovary, prostate, nerve, cartilage, bone, brain, or portion thereof.

The gel of the filler can include polymeric materials which are capable of forming a hydrogel may be utilized. In one embodiment, the polymer forms a hydrogel within the body. A hydrogel is defined as a substance formed when an organic polymer (natural or synthetic) is cross-linked via covalent, ionic, or hydrogen bonds to create a three-dimensional open-lattice structure which entraps water molecules to a gel. Naturally occurring and synthetic hydrogel forming polymers, polymer mixtures, and copolymers may be utilized as hydrogel precursors.

In some aspects, the hydrogel can be formed by a composition formed by mixing constituents together (E.g., accelerant fluid, diluent, and PEG together) and may comprise one or more polysaccharide compounds or a salt thereof. For example, the composition may include a cellulose compound such as carboxymethyl cellulose (CMC) or salt thereof (e.g., CMC) sodium, xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be cross-linked with a suitable crosslinking compound, such as butanediol diglycidyl ether (BDDE). In some aspects, the polysaccharide may be a homopolysaccharide or a heteropolysaccharide

The present disclosure also provides mixing systems to form the gel composition and corresponding medical devices for use and/or delivery to a treatment site of a patient. According to some aspects of the present disclosure, the mixing system may include a plurality of reservoirs with respective lumens. Collectively, the lumens therein may serve as a container for constituents to mix the gel composition of this disclosure. Suitable reservoirs may include, for example, syringes (e.g., a syringe barrel compatible with a manual or automatic injection system) and other fluid containers configured for use with a suitable injection needle. Exemplary materials suitable for the reservoir include, but are not limited to, cyclic olefin polymer, polypropylene, polycarbonate, polyvinyl chloride, and glass. In some aspects, one of these materials (e.g., cyclic olefin copolymer specifically) can have a coating applied to it, such as SiO₂), which is advantageous so the coating can perform as a primary oxygen barrier, behave as a glass-like layer, and can be applied using a vapor deposition process.

According to some aspects of the present disclosure, the compositions may include at least one accelerant (e.g., an activating agent) combined with a precursor mixed from a diluent (e.g., mostly water) and polyethylene glycol (PEG). In some examples, the composition may be or include a gel with a desired gel strength and/or viscosity, such as a biocompatible gel suitable for injection (e.g., through a needle).

The hydrophilic polymer can be any gelling agent(s), including natural ones or synthetic in origin, and may be anionic, cationic, or neutral. Non-limiting examples of the gelling agents include polysaccharides such as gellan gum, xanthan gum, gum arabic, guar gum, locust bean gum, alginate, and carrageenans.

The concentrations of gelling agent(s) in the composition described in this disclosure may range from about 0.01% to about 2.0% by weight with respect to the total weight of the composition, such as from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, the composition may have a viscosity ranging from about 0.001 Pascal-second (Pa·s) to about 0.100 Pa·s at a shear rate of 130 s⁻¹, such as, e.g., from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, the composition may have a viscosity ranging from about 0.001 Pa·s to about 0.050 Pa·s at a shear rate of 768 s⁻¹, such as, e.g., from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹ .In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

The mixing system herein may include or be removably connected to one or more needles. In some examples, the needle may be a hypodermic needle, and may range from a size of 7 gauge (4.57 mm outer diameter (OD), 3.81 mm inner diameter (ID)) to 33-gauge (0.18 mm OD, 0.08 mm ID), e.g., a size of 16 gauge (1.65 mm OD, 1.19 mm ID), 18 gauge, 21 gauge (0.82 mm OD, 0.51 mm ID), 22 gauge (0.72 mm OD, 0.41 mm ID), 23 gauge (0.64 mm OD, 0.33 ID), or 24 gauge (0.57 mm OD, 0.31 mm ID). Exemplary materials for the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

According to some aspects of the present disclosure, the filler compositions herein, e.g., the compositions prepared by the methods herein may have sufficient strength, e.g., gel strength, to withstand the forces and thus minimizing the effects of the forces on the continuity of the three-dimensional gel network. In the meantime, the composition with sufficient strength may have a viscosity suitable for injection, e.g., a viscosity that does not render the composition stuck in the reservoir(s), delivery lumen, or a needle connected therewith.

According to some aspects of the present disclosure, the composition may maintain its three-dimensional structure until the gel is injected through a needle, whereupon the structure may form fragments of the original continuous, three-dimensional network. Those gel fragments may have a diameter corresponding to the diameter of the injection needle, such that the fragments are as large as possible in-vivo to retain as much of the three-dimensional structure of the gel as possible. Injection of these larger-sized particles or fragments is believed to increase the amount of time the gel remains within the tissue.

The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 lbf to about 25 lbf, such as from about 10 lbf to about 20 lbf, e.g., about 15 lbf. The loads measured for a given gel concentration may vary for different needles and flow rates.

According to some aspects of the present disclosure, the size of the needle may be chosen based on the viscosity and/or components of the composition, or vice versa. According to some aspects of the present disclosure, the size of the needle may be 23 gauge or 25 gauge. In some cases, a larger size of 18-gauge, 20 gauge, 21 gauge, or 22 gauge may be used to inject the compositions herein.

According to some aspects of the present disclosure, the mixing system of this disclosure can be included in a kit for introducing a filler into a patient, whereby the filler can include any of the gel compositions of this disclosure. Kits or systems for mixing a gel composition of this disclosure, such as hydrogels, may be prepared so that the precursor(s) and any related activating agent(s) are stored in the kit with diluents as may be needed. Applicators may be used in combination with the same. The kits can be manufactured using medically acceptable conditions and contain components that have sterility, purity and preparation that is pharmaceutically acceptable. Solvents/solutions may be provided in the kit or separately. The kit may include syringes and/or needles for mixing and/or delivery. The kit or system may comprise components set forth herein.

During some examples of use, once saline has been injected to the treatment site, a mixing system can be connected to a needle (e.g., an 18-gauge spinal needle) to then inject a 5-10 mm layer of filler (e.g., gel composition) along the posterior wall of the prostate between the prostate and rectum. Once the filler has been injected into the space between the rectum and prostate, ultrasound images can be obtained.

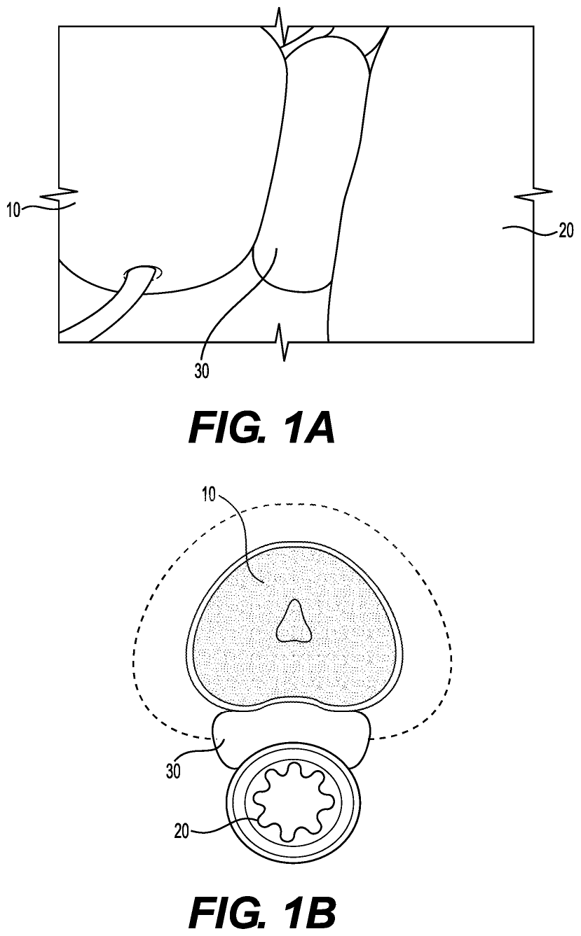

Turning to the drawings, FIG. 1A is a perspective view and FIG. 1B is a partial cross-section view illustrating example filler 30, in the form of a gel composition having been delivered by the mixing system of this disclosure between rectum 20 and prostate 10 of a patient in Denonvilliers' space.

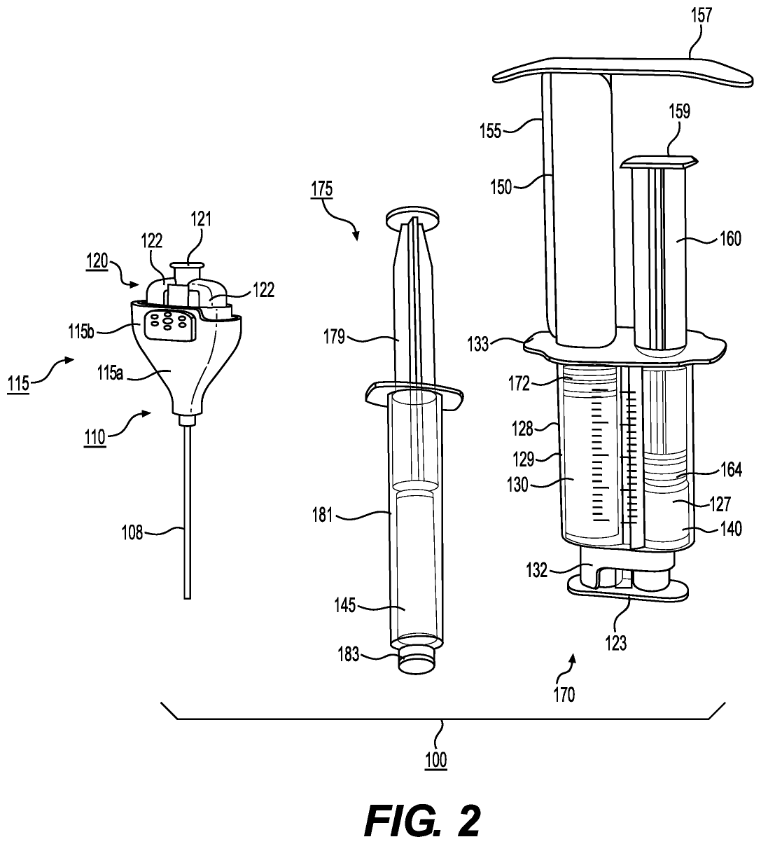

FIG. 2 shows an exploded view of an exemplary mixing system 100 in accordance with certain aspects of the present disclosure for mixing a gel composition for use as filler 30. The system 100 can be packaged in a kit and include a syringe 175 with a fluid (e.g., diluent), a main assembly 170, and a needle assembly 110 attachable to the main assembly 170. Syringe 175 can include a plunger rod 179 with an upper flange surface configured so a user can advance rod 179 and flush a constituent 145 (e.g., a fluid such as diluent) from syringe 175 out through port 183. As used herein, the term “fluid” is defined broadly and can include liquids, gels and particulate matter such as granules, pellets, or powders, or any combination of liquids, gels, oils, and/or particulate matter (e.g., granules, pellets, or powders).

Needle assembly 110 can include needle 108, which can be any needle of this disclosure suitable for hydrodissection as well as delivering the gel composition (e.g., filler 30) to the treatment site. A proximal end of needle 108 can be connected to a distal end of a connector 115 with asymmetric coupling portion 115b. Connector 115 can include a distal, symmetric portion 115a opposite portion 115b that is configured to connect with the main assembly 170 of system 100 or adaptor 120.

Main assembly 170 of system 100 can include a multi-lumen chamber formed by a first lumen 127 inside a first barrel and a second lumen 129 inside a second barrel. Each lumen 127, 129 can be oriented parallel with the other, running side-by-side. A plunger stopper 164 can be located at a distal end of a first plunger rod 160. Rod 160 can be advanceable within lumen 127. Rod 160 can be advanced by flange 159 positioned on a proximal end of rod 160. In some examples, flange 159 is shaped and arranged so that it is prevented from advancing proximally past flange 157.

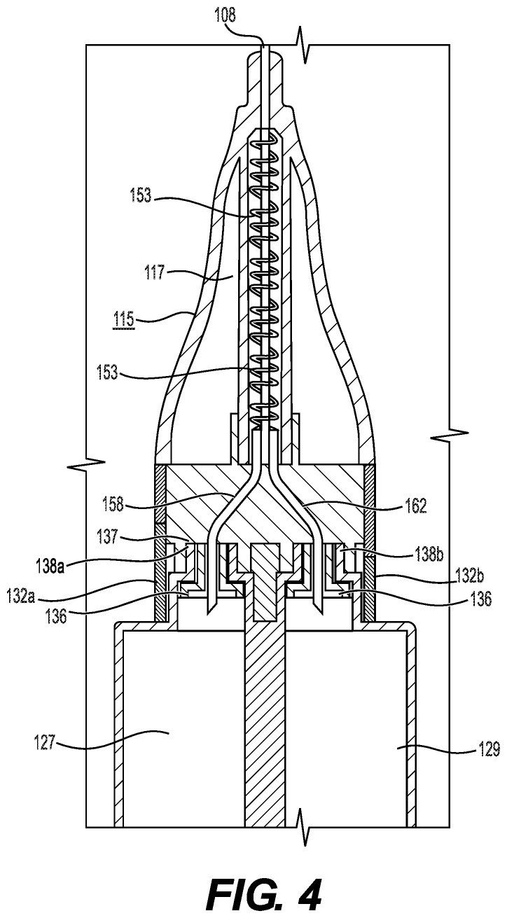

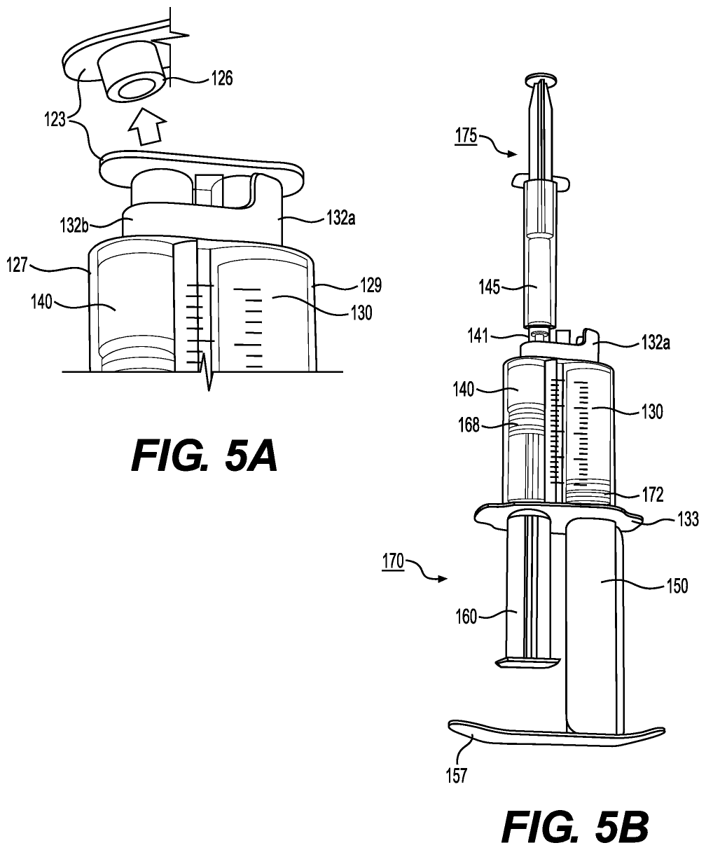

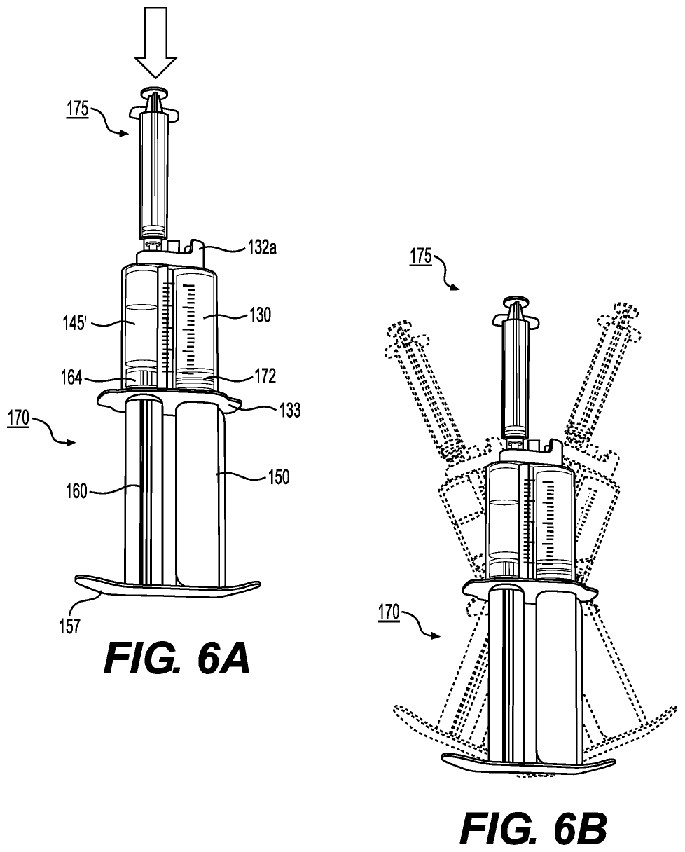

Lumen 127 can include one or more constituents (e.g., a fluid, liquid or otherwise). As desired, distally moving rod 160 can cause stopper 164 to advance constituent(s) out from lumen 127 through ports 138a, as shown more clearly in FIGS. 4 and 5B. In some examples, at least one of the constituents of lumen 127 can include constituent 140 (e.g., an activating agent, such as PEG or any other agent mixable with constituent 145 to intermix and form precursor 145′. The diluent used in systems of this disclosure, including system 100, can be a branched polymer having a plurality of succinimidyl termini dissolved in a low pH (4.0) containing a low molecular weight precursor comprising nucleophiles, though other diluent fluid solutions are contemplated within the scope of this disclosure. Once constituent 140, 145 are mixed together, precursor 145′ can be formed in portion 127b, as shown in FIG. 6A.

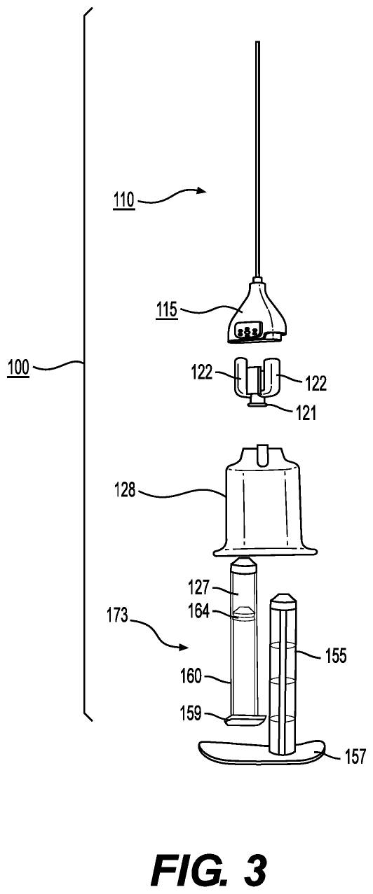

Turning back to FIG. 2, lumen 129 can similarly include a plunger rod 155 slidable therein. A distal end of rod 155 can include a stopper 172. A proximal end of rod 155 can include an actuating flange 157 configured so that a user can a press thereon to drive rod 155 proximally or distally. As seen clearly in FIG. 3, plunger rod 155, flange 157, rod 160, and lumen 127 can alternatively be partially or entirely integrally formed together to form plunger assembly 173. Assembly 170 can be assembled to form lumen 129 by positioning distal ends of rods 155, 160 with distal ends of receiver 128. Receiver 128 can include an open proximal end with a flange 133 while the distal ends of receiver 128 can include plurality of smaller openings configured to receive ports 138a, 138b of assembly 170. This is shown more clearly in FIG. 3, which illustrates an exploded view of system 100, including plunger assembly 173 proximal and just prior to being assembled through the open upper end of receiver 128.

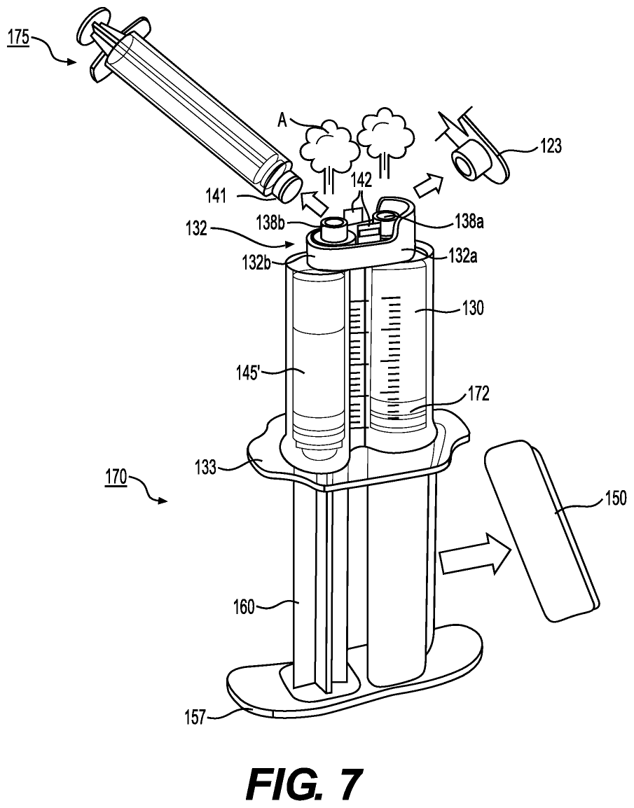

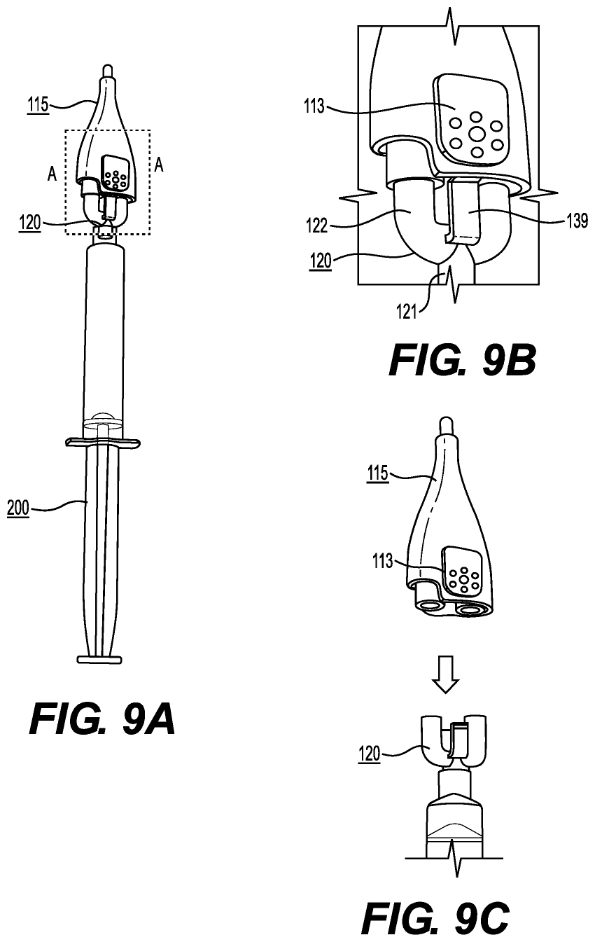

Each of ports 138a, 138b are configured to couple to corresponding receivers of connector 115 and permit egress of fluids from respective lumens 127, 129 into connector 115. It is noted that aspects of adaptor 120, shown in FIG. 2 coupled with connector 115, is described more particularly in FIGS. 9A-9C. Adaptor 120 is an aspect of this disclosure separate from main assembly 170. The shape and position of ports 138a, 138b are clearly shown in FIG. 7. In some aspects, port 138b can be positioned on one side of asymmetric coupler 132, for example portion 132b, which as shown in FIG. 7 can include a shape that differs from portion 132a of coupler 132 opposite thereof. In this respect, portion 132a can be shaped different from portion 132b so that port 138a can only couple to a corresponding receiver side of connector 115. As can be seen in FIGS. 2-4, portion 115b of connector 115 is asymmetric with one side higher than the other and portions 132a, 132b of coupler 132 form a mirror shape of portion 115b. The shape of coupler 132 and portion 115b can be any shape so long as it is asymmetric to guide connector 115 and coupler 132 into the specific orientation and arrangement so that ports 138a, 138b are connectable in fluid communication with corresponding receivers (e.g., tubes 158, 162) of connector 115.

Preferably, portion 132a is higher than portion 132b so that syringe 175 is incapable of inserting constituent 145 into port 138a of adjacent portion 132a. Instead, portion 132b being lower allows the distal end of syringe 175 to couple with port 138b, which is adjacent the lower portion 132b. Also optionally, in a first state before mixing, the system 100 can include a retainer 150 removably positioned between flanges 133 and 157 so as to prevent unwanted movement rod of 155. In some aspects, once assembly 173 is nested within receiver 128, lumen 129 can be formed. Constituent 130, such as an accelerant, can be positioned within lumen 129, as shown clearly in FIG. 2, and stopper 172 of rod 155 can advance constituent 130 to be distal of ports 138a, 138b and mix with precursor 145′. In some aspects, while flange 157 is permanently or temporarily attached to the button of flange 159 of rod 160, distally advancing flange 157 can distally advance both stopper 172 as well as rod 160, and stopper 164, so that the precursor 145′ and constituent 130 are capable of egressing through respective ports 138 and mixing together distal constituent 130 thereof (e.g., in connector 115) to form the mixture (e.g., the gel composition). In some aspects, once flanges 157, 159 are aligned or otherwise attached, flange 157 being distally advanced can drive both rod 160 and rod 155 simultaneously.

Turning back to needle assembly 110, it can be seen that connector 115 includes portions 115a, 115b with lower, asymmetric shape to couple to coupler 132. Portion 115b can be relatively solid, rather than relatively hollow, and insertable into an open proximal end of portion 115a to nest therewith and form connector 115. Portion 115a can be substantially hollow with a tapered or Y-shape profile for its outer surface. Portion 115a can terminate in a distal end with a central lumen 117 running therethrough. In some aspects, lumen 117 can be a mixing lumen for one or more constituents of system 100 to mix. In some aspects, lumen 117 can be integrally formed with system 100, rather than be a feature of connector 115.

As seen more clearly in FIG. 4, which is a partial cross-section view of connector 115 assembled with a distal end of assembly 170, each of lumens 127, 129 can be in fluid communication with a proximal end of lumen 117 of connector 115. Portion 115b can be asymmetric and coupled to coupler 132 with a fluid path 137 running from ports 138a, 138b to a proximal end of lumen 117. Lumen 117 can include a static mixer 153 so that fluid from respective lumens 127, 129 can mix together and form the gel composition to be delivered through needle 108.

In some aspects, portion 115b can include tube 158 (e.g., a hypotube) with a proximal end configured in fluid communication with lumen 127 and pierce a corresponding membrane or seal 136 of ports 138a, 138b. Portion 115b can also include a tube 162 (e.g., a hypotube) with a proximal end configured in fluid communication with lumen 129 and pierce a corresponding membrane or seal 136 of port 138. In this respect, once precursor 145′ is in position in lumen 127 and constituent 130 is positioned in lumen 129 with air purged from each and connector 115 assembled thereto, distally moving rod 155 can cause precursor 145′ and constituent 130 to egress through respective ports 138 and respective tubes 158, 162 to mix with each other in lumen 117. Tubes 158, 162 can form a Y-shape, as in FIG. 4, though any other shape can be used as needed or required.

Now, turning to FIGS. 5A-8B are example steps of a process of using system 100 according to certain aspects of this disclosure. While certain steps are shown as a sequence between each figure, in other embodiments, fewer steps are contemplated and the order by which steps are performed can be different than what is illustrated. In FIG. 5A, system 100 is introduced and a cap 123, including each of its receivers 126, can be detached from ports 138a, 138b. Cap 123 can be configured to seal ports 138a, 138b between uses or during transit when stored in separate packaging or a kit. Lumen 127 can include constituent 140 (e.g., a hydrophilic polymer, PEG, etc.,) whereas lumen 129 can include constituent 130.

In FIG. 5B, system 100 is illustrated in a first state with rod 155 fully retracted and retainer 150 positioned between flanges 133, 157. In the depicted configuration, rod 155 is incapable of distally moving as a result of retainer 150 being wedged between flanges 133, 157. In contrast, precursor 145′ is capable of being mixed by the depicted coupling of syringe 175 to port 138b. Syringe 175 can include luer fitting 141 at a distal end so that syringe 175 can be securely engaged with port 138b. The asymmetric shape of coupler 132 prevents fitting 141 of syringe 175 from coupling to port 138a. In the illustrated state, rod 179 is fully withdrawn and capable of advancing constituent 145 from syringe 175 into assembly 170. In FIG. 6A, rod 179 has been advanced towards ports 138a, 138b thereby injecting constituent 145 into lumen 127 so that constituent 140, 145 can mix together and form precursor 145′.

In FIG. 6B, assembly 170 and syringe 175 can be shaken back and forth to ensure precursor 145′ forms as a result of mixing between constituent 140, 145, while constituent 130 remains in lumen 129. Preferably, the shaking action of FIG. 6B is done while the ports 138a, 138b are oriented generally upward. While syringe 175 is illustrated during the act of shaking in FIG. 6B, in other examples syringe 175 can be detached from assembly 170 prior to shaking. Further, the shaking to effect proper mixing of precursor 145′ can be performed in other orientations (e.g., generally downward, etc.), as needed or required.

In FIG. 7, with precursor 145′ formed and constituent 130 in lumen 129, syringe 175 and retainer 150 can be removed from assembly 170. To the extent a cap (e.g., cap 123) is still connected to port 138a, it too can be removed. Flange 157 can now be distally advanced slightly to purge any air A from system 100 out through respective ports 138a, 138b. Preferably, air A is purged as shown while system 100 is oriented generally upward.

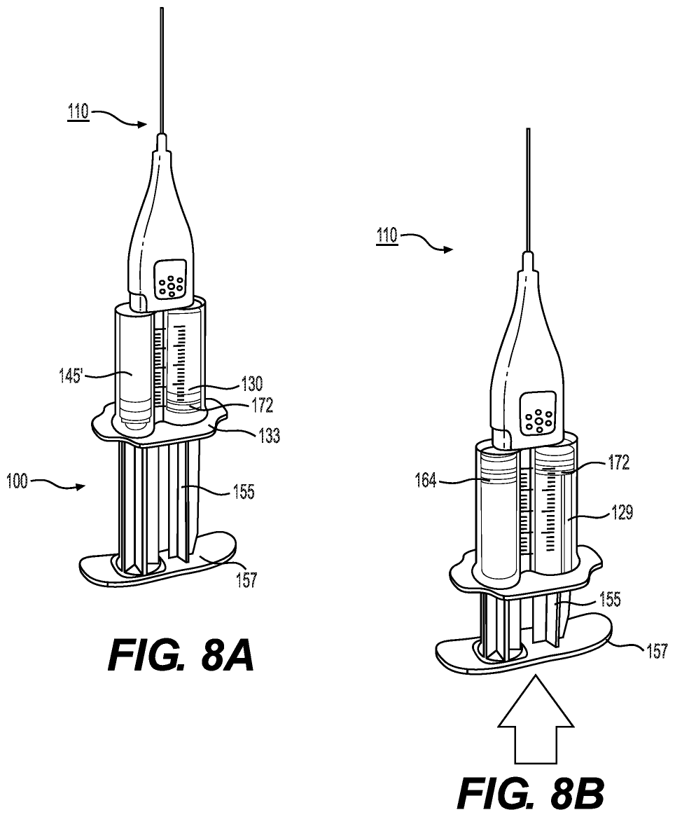

Turning to FIG. 8A, system 100 is now connected to connector 115, which is primed and connected to needle 108 in position at the treatment site. Aspects of priming connector 115 are discussed more in FIGS. 9A-9C. In FIG. 8B, a user can advance flange 157 distally, as denoted by the upward arrow, so that corresponding rods 155, 160 distally drive respective stoppers 164, 172 and advance precursor 145′ from lumen 127 and constituent 130 from lumen 129, through ports 138, and into the central lumen 117 of connector 115. As long as flange 157 continues advancing, precursor 145′ and constituent 130 can mix within central lumen 117 and continue egressing through needle 108 and ultimately to the treatment site. Optionally, lumen 117 can include the static mixer 153 configured to thoroughly mix the fluids together to form the gel composition to be delivered to the treatment site. System 100 as shown is relatively easy to assemble and minimizes potential unintentional gel mixing errors prior to delivery.

Separately, in FIG. 9A, connector 115 is shown connected to syringe 200 via adaptor 120, as illustrated more clearly in FIG. 9B which is a close-up of section A-A. Adaptor 120 can provide a fluid bridge 122 from each of tubes 158, 162 of connector 115 to syringe 200 via tubular end 121. Connector 115 can include an externally positioned button 113 to open and close corresponding connecting latches of connector 115. In this respect, adaptor 120 can include one or more latches 139 configured to removably attach to connector via button 113. Button 113 can similarly be used to removably connect to latches 142 of coupler 132 when engaging connector 115 with assembly 170.

Turning back to FIGS. 9A-9C, adaptor 120 can couple to syringe 200 with a luer fitting (e.g., end 121) or any other connector operable to connect with a distal end of syringe 200. While not shown, during use connector 115 is contemplated to be connected to needle 108 for hydrodissection at the treatment site with saline from the syringe 200. After hydrodissection with syringe 200, needle 108 and adaptor 120 can be released from connector 115, as shown in FIG. 9C. However, other coupling approaches between connector 115, adaptor 120, and syringe 200 are contemplated as needed or required.

For example and without limitation, snap fit connectors, magnetic connectors, female-male connectors, hook and loop fasteners and the like are contemplated.

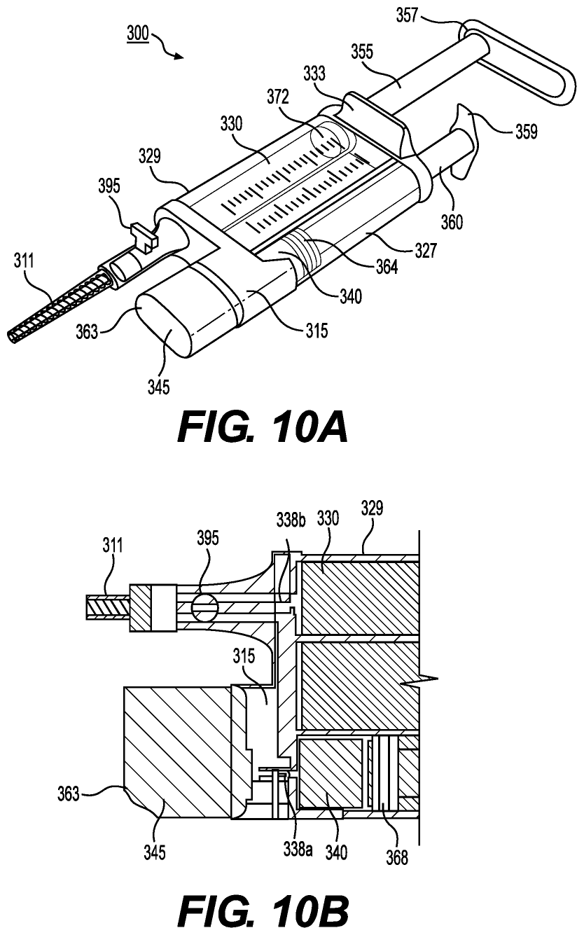

FIG. 10A shows a perspective view of another exemplary mixing system 300 in accordance with certain aspects of the present disclosure for mixing a mixture, such as a gel composition for use as filler 30. The system 300 can be packaged in a kit and include a multi-lumen chamber formed by first lumen 327 inside a first barrel and a second lumen 329 inside a second barrel. Each lumen 327, 329 can be oriented parallel with the other, running side-by-side, which in system 300 can be a single integrated structure with lumens 327, 329 separately formed therein. A plunger stopper 364 can be located at a distal end of a first plunger rod 360. Rod 360 can be advanceable within lumen 327. Rod 360 can be advanced by flange 359 positioned on a proximal end of rod 360. In some examples, flange 359 is shaped and arranged so that it is prevented from advancing proximally past flange 357.

Lumen 327 can include one or more constituents (e.g., a fluid, liquid or otherwise). As desired, distally moving rod 360 can cause stopper 364 to advance constituent(s) out from lumen 327 through port 338a, as shown more clearly in close-up of FIG. 10B. In some examples, lumen 327 can include constituent 340, such as PEG or any other agent mixable with diluent to form precursor. Distal of lumens 329, 327 can be connector 315 which can include a fluid chamber 363. Chamber 363 may include one or more constituents (e.g., a fluid, liquid or otherwise), such as constituent 345 in fluid communication with constituent 340 in lumen 327 via port 338a. In this respect, rod 360 can be retracted so that constituent 345 can be withdrawn from chamber 363 through port 338a and into lumen 327 to mix with constituent 340 to form precursor 345′. Chamber 363 can be integrally formed with connector 315 or detachable therewith.

Lumen 329 can similarly include a plunger rod 355 slidable therein. A distal end of rod 355 can include a stopper 372. A proximal end of rod 355 can include an actuating flange 357 configured so that a user can a press thereon to drive rod 355 proximally or distally. Lumen 329 can terminate in a port 338b through which fluid of lumen 329 can flow. In some examples, a mixing lumen 311 can be provided in fluid communication with both port 338a, 338b so that fluids from lumens 327, 329 can be directed therethrough before reaching needle 308. Lumen 311 can include valve 395 actuatable by user. As shown in FIGS. 10A and 10B, valve 395 can be twistable or rotatable between flow position (FIG. 10B) and flow closed position (FIG. 10A). In some examples, flange 357 may be larger than flange 359 so that flange 359 is configured to contact flange 357 and prevented from advancement therepast. In some examples, flanges 357, 359 can be oriented orthogonal with the other to further induce contact between the respective flanges and thereby prevent advancement of flange 359 passed flange 357.

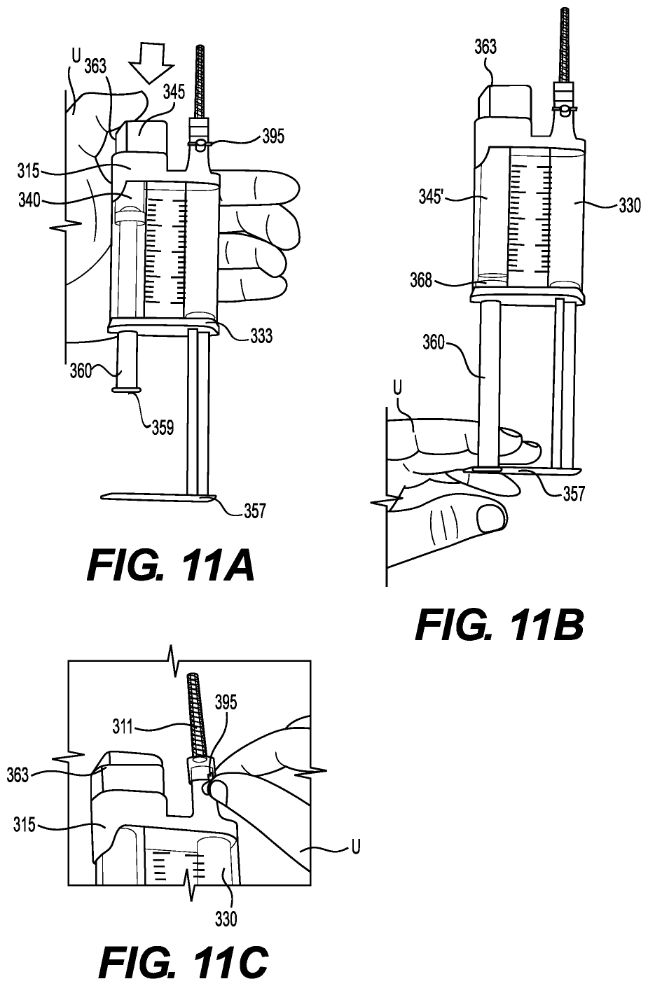

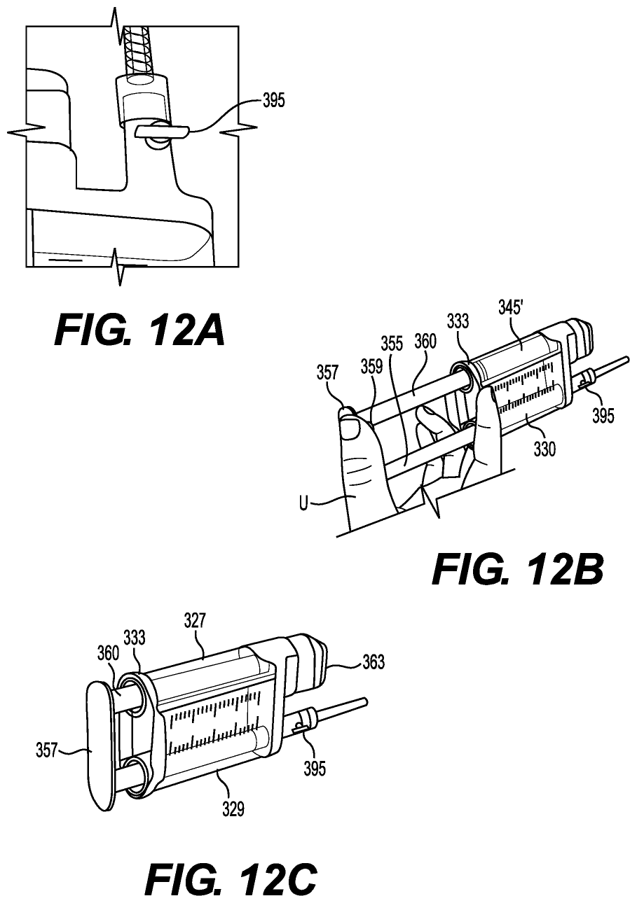

FIGS. 11A-12C show example steps of a process of using system 300 according to certain aspects of this disclosure. While certain steps are shown as a sequence between each figure, in other embodiments, fewer steps are contemplated and the order by which steps are performed can be different than what is illustrated. In FIG. 11A, chamber 363 is shown being attached by user U to connector 315 while valve 395 is denoted in the closed position. In FIG. 11B, rod 360 has been withdrawn thereby causing constituent 345 previously housed in chamber 363 to be drawn into lumen 327 to mix and form precursors 345′. In some examples, all of constituent 345 is known to be in lumen 327 when flange 359 is adjacent or in contact with flange 357. In FIG. 11C, user U rotates valve 395 so as to open the fluid path in lumen 311. Now, any fluids from system 300 can flow into lumen 311, be mixed, and then delivered through needle 108 (not depicted). FIG. 12A merely shows an example valve 395 in a closed position. For fluid to flow through lumen 311 from lumens 327, 329, valve 395 needs to be open, as in FIG. 12B-FIG. 12C, where the user U is shown beginning to (FIG. 12B) and then advancing (FIG. 12C) flanges 357, 359 distally so as to cause corresponding stoppers 364, 372 to move constituents 345′, 330 through respective ports 338a, 338b and into lumen 311.

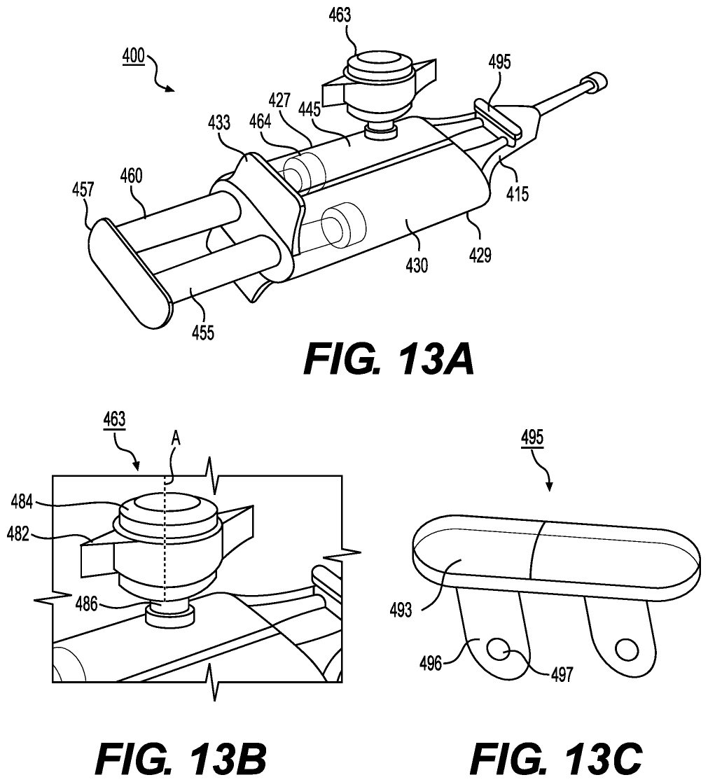

FIG. 13A shows a perspective view of another exemplary mixing system 400 in accordance with certain aspects of the present disclosure for mixing a mixture, such as a gel composition for use as filler 30. The system 400 can be packaged in a kit and include a multi-lumen chamber formed by first lumen 427 inside a first barrel and a second lumen 429 inside a second barrel. System 400 is similar to previous system 300, except for chamber 463 is now extended directly from a side of lumen 427. Chamber 463 is shown more particularly in FIG. 13B and can be a rotatable housing with an outer portion 484, twisting arms 482, and port 486 extended directly from an outer surface of lumen 427. Arms 482 can be grasped or otherwise rotated by user so as to open or close a corresponding valve and initiate flow of constituents housed in chamber 463 into lumen 427. In some examples, lumen 427 can be pre-loaded with constituent 445 (e.g., diluent) and chamber 463 can include constituent 440 (e.g., PEG), so that twisting arms 482 a first direction or first rotation open or close a corresponding valve so fluid of chamber 463 can flow into lumen 427 and mix to form precursor 445′, similar to previous mixing systems of this disclosure.

System 400 can also include valve insert 495, as shown in FIGS. 13A and 13C. Valve insert 495 can be detachable from connector 415. Connector 415 can include one or more inner lumens through which constituents from lumens 427, 429 can be received. However, flow in the lumen(s) of connector 415 can be effectively blocked if valve insert 495 is absent. As can be seen in FIG. 13C, valve insert 495 can include an upper, planar surface 493 with one or more extensions 496. Each extension 496 can include an opening 497 through which fluid can flow between corresponding lumen of connector 415 when insert 495 is coupled therewith. In some examples, at least one membrane or seal can be at or adjacent the opening of connector 415 through which valve insert 495 is positioned. In this respect, the seal or membrane can remain closed when valve insert 495 is absent but opened to permit fluid flow when insert 495 is present. The example mixing systems of this disclosure are merely for illustrative purposes, and other orientations, features, and shapes of are contemplated as needed or required, within the scope of this disclosure.

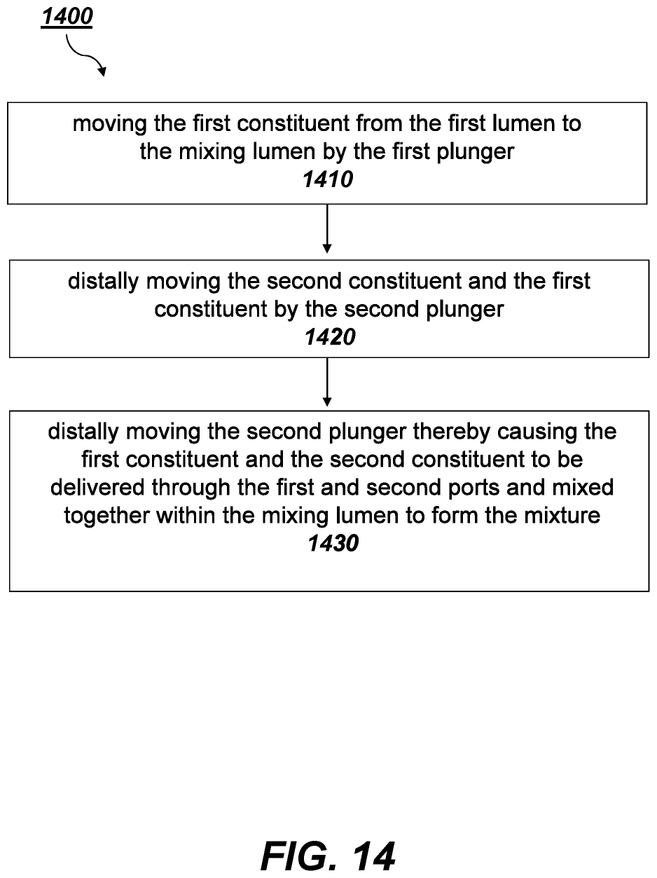

FIG. 14 depicts a method 1400 of using any of the herein disclosed mixing systems. Step 1410 of method 1400 can include moving the first constituent from the first lumen to the mixing lumen by the first plunger. Step 1420 of method 1400 can include distally moving the second constituent and the first constituent by the second plunger. Step 1430 of method 1400 can include distally moving the second plunger thereby causing the first constituent and the second constituent to be delivered through the first and second ports and mixed together within the mixing lumen to form the mixture. Method 1400 can end after step 1430. In other embodiments, additional steps according to the examples described above can be performed.

The systems and methods of this disclosure are beneficial by reducing the number of system components, are relatively simply to assemble and operate, with minimal mixing errors prior to delivery within a patient at a treatment site. Other aspects and embodiments of the present disclosure will be apparent to those skilled in the art from consideration of the specification and practice of the embodiments disclosed herein. While certain features of the present disclosure are discussed within the context of exemplary procedures, the compositions, systems, and methods may be used for other medical procedures according to the general principles disclosed. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein. It is intended that the specification and examples be considered as exemplary only, with a true scope and spirit of the present disclosure being indicated by the following claims.

What is claimed is:

1. A system for producing a mixture to deliver to a treatment site, comprising:
a mixing lumen attachable to a proximal end of a delivery system;
a multi-lumen chamber connected to a proximal end of the mixing lumen, the multi-lumen chamber comprising a first lumen aligned and adjacent a second lumen;
the first lumen configured to receive a first constituent in a first state, the first constituent being insertable through a first port into the first lumen, the first lumen comprising a first plunger to move the first constituent from the first lumen to the mixing lumen; and
the second lumen configured to comprise a second constituent, the second lumen comprising a second plunger to distally move the second constituent and the first constituent in a second state, and the second lumen terminating in a second port;
wherein in the second state, distally moving the second plunger causes the first constituent and the second constituent to be delivered through the first and second ports and mixed together within the mixing lumen to form the mixture.

2. The system of claim 1, wherein the first port comprises a luer fitting configured to receive a distal end of a syringe, and wherein the distal end of the syringe and the first port are configured to removably connect with each other.

3. The system of claim 1, wherein the first port is configured so that air can be purged from the first lumen through the first port.

4. The system of claim 1, wherein the mixing lumen is comprised in a connector, the connector comprising a first asymmetric coupler positioned on a proximal end of the connector; and
wherein the multi-lumen chamber comprises a second asymmetric coupler positioned on a distal end of the multi-lumen chamber and configured to removably connect to the first asymmetric coupler.

5. The system of claim 4, the connector further comprising at or adjacent the first asymmetric coupler;
a first tube configured to be in fluid communication with the first port and the mixing lumen in the second state; and
a second tube configured to be in fluid communication with the second port and the mixing lumen in the second state.

6. The system of claim 4, wherein the first asymmetric coupler comprises one or more latches configured to removably connect with one or more latches of the second asymmetric coupler,
wherein at least one of the first and second asymmetric coupler comprise an actuator positioned on an outer surface configured to be squeezed so as to release a coupling between the one or more latches of the first and second asymmetric couplers.

7. The system of claim 4, wherein the first and second asymmetric couplers each comprise an asymmetric outer surface profile shaped as a mirror of the other.

8. The system of claim 1, wherein the first lumen comprises a hydrophilic polymer prior to injection of the first constituent into the first lumen.

9. The system of claim 1, wherein injecting the first constituent through the first port into the first lumen by a syringe causes the first plunger to move proximally so the first lumen so the first constituent is received in the first lumen in the first state.

10. The system of claim 1, wherein once the first and second constituents are comprised within the first and second lumens, respectively, the second plunger is configured to distally move a first distance to purge air from the multi-lumen chamber.

11. The system of claim 1, wherein the mixture is a hydrogel.

12. The system of claim 1, wherein in a first state, the system further comprises a retainer removably positioned between a flange of the second plunger and the multi-lumen chamber so as to prevent movement of the second plunger; and
wherein in the second state, the retainer is removed so that the second plunger is capable of moving in order to distally move the second plunger to cause at least one of the first constituent and the second constituent and mix together in the mixing lumen to form the mixture.

13. A method for producing a mixture by a mixing system, the mixing system comprising a multi-lumen chamber connected to a proximal end of a mixing lumen, the multi-lumen chamber comprising a first lumen aligned and adjacent a second lumen, the first lumen configured to receive a first constituent in a first state, the first constituent being insertable through a first port into the first lumen,
the first lumen comprising a first plunger to move the first constituent from the first lumen to the mixing lumen; and
the second lumen comprising a second constituent, a second plunger, and the second lumen terminating in a second port, the method comprising:
moving the first constituent from the first lumen to the mixing lumen by the first plunger;
distally moving the second constituent and the first constituent by the second plunger; and
distally moving the second plunger thereby causing the first constituent and the second constituent to be delivered through the first and second ports and mixed together within the mixing lumen to form the mixture.

14. The method of claim 13, wherein the first port comprises a luer fitting configured to receive a distal end of a syringe, and wherein a distal end of the syringe and the first port are configured to removably connect with each other.

15. The method of claim 13, further comprising: purging air from the first and second lumens by distally moving the second plunger a first distance before forming the mixture.

16. The method of claim 15, wherein the step of purging comprises egressing air through an air-permeable fluid-impermeable membrane positioned on at least one of the first port and the second port.

17. The method of claim 13, wherein the mixing lumen is comprised in a connector comprising a first asymmetric coupler positioned on a proximal end of the connector;
wherein the multi-lumen chamber comprises a second asymmetric coupler positioned on a distal end of the multi-lumen chamber and configured to removably connect to the first asymmetric coupler;
wherein the first asymmetric coupler comprises one or more latches configured to removably connect with one or more latches of the second asymmetric coupler,
wherein at least one of the first and second asymmetric coupler comprise an actuator positioned on an outer surface configured to be squeezed so as to release a coupling between the one or more latches of the first and second asymmetric couplers.

18. The method of claim 13, wherein the mixing lumen is comprised in a connector comprising a first asymmetric coupler positioned on a proximal end of the connector;
wherein the multi-lumen chamber comprises a second asymmetric coupler positioned on a distal end of the multi-lumen chamber and configured to removably connect to the first asymmetric coupler,
wherein the first and second asymmetric couplers each comprise an asymmetric outer surface profile shaped as a mirror of the other.

19. The method of claim 13, further comprising:
connecting a primed connector and a delivery system to an asymmetric coupler of the multi-lumen chamber, the primed connector comprising the mixing lumen.

20. The method of claim 13, further comprising:
positioning a hydrophilic polymer in the first lumen prior to injection of the first constituent into the first lumen.
